(19) United States
(12) Patent Application Publication
(10) Pub. No.: US 2015/0265810 A1
(43) Pub. Date: Sep. 24, 2015

(54) GUIDEWIRE

(71) Applicant: ASAHI INTECC CO., LTD., Nagoya (JP)

(72) Inventor: Yumiko NAKAGAWA, Seto-shi (JP)

(21) Appl. No.: 14/657,051

(22) Filed: Mar. 13, 2015

(30) Foreign Application Priority Data
Mar. 19, 2014 (JP) 2014-055799

(51) Int. Cl. A61M 25/09

(52) U.S. Cl. CPC A61M 25/09; A61M 2025/09083

(57) ABSTRACT

A guidewire includes a core shaft and a first coil body that is wound around a distal end portion of the core shaft. In the guidewire, a second coil body is joined to a proximal end portion of the core shaft by a joint, the second coil body including a stranded wire composed of a plurality of wires that are twisted together.

### CROSS REFERENCE TO RELATED APPLICATION

This application claims priority to Japanese Patent Application No. 2014-055799 filed in the Japan Patent Office on Mar. 19, 2014, the entire contents of which are incorporated herein by reference.

### BACKGROUND

The disclosed embodiments relate to a guidewire that is used as a medical device that is inserted into a body cavity for the purpose of treatment or examination.

Hitherto, various guidewires have been proposed as guidewires for guiding, for example, a catheter that is used by being inserted into body tissues or tubular organs, such as blood vessels, the alimentary canal, or ureters, for performing treatment or examination.

For example, Japanese Unexamined Patent Application Publication No. 2007-90097 discloses a guidewire including a shaft whose diameter gradually increases towards a proximal end side and a coil that is wound around a tip portion of the shaft. In the guidewire, an intermediate portion of the coil is secured to the shaft with a securing material.

### SUMMARY

Here, for example, when inserting the guidewire that is discussed in Japanese Unexamined Patent Application Publication No. 2007-90097 (that is, the guidewire including a shaft whose diameter gradually increases towards the proximal end side) along an inverted U-shaped path extending from a blood vessel in the right leg to a blood vessel in the left leg by a cross over method, in addition to the operability of the guidewire being reduced due to a difficulty in following the blood vessels in the legs that are considerably bent, the shaft may be excessively bent due to a load that is received by the shaft when the shaft, for example, contacts vascular walls. As a result, for example, permanent deformation occurs in a proximal end side (large-diameter portion) of the shaft. This may hinder subsequent operations of the guidewire.

Accordingly, it is an object of the disclosed embodiments to provide a guidewire that, even when the guidewire is inserted into a blood vessel that is considerably bent, is capable of ensuring high followability and is capable of reducing permanent deformation.

To this end, according to a first aspect of the disclosed embodiments, there is provided a guidewire including a core shaft and a first coil body that is wound around a distal end portion of the core shaft. In the guidewire, a second coil body is joined to a proximal end portion of the core shaft by a joint, the second coil body including a stranded wire that includes a plurality of wires that are twisted together.

According to a second aspect of the disclosed embodiments, there is provided a guidewire including a second coil body and a first coil body that is wound around a distal end portion of the second coil body. In the guidewire, the second coil body includes a stranded wire that includes a plurality of wires that are twisted together.

In the guidewire according to the first aspect, the second coil body is joined to the proximal end portion of the core shaft by a joint, the second coil body including a stranded wire that includes a plurality of wires that are twisted together. In such a stranded wire (the second coil body), the wires can move slightly relative to each other. Therefore, in addition to the degree of freedom and the flexibility being high, sufficient restoring force is also ensured.

Therefore, for example, when inserting the guidewire along an inverted U-shaped winding path extending from a blood vessel in the right leg to a blood vessel in the left leg by a cross over method, it becomes possible to easily follow the shapes of blood vessels that are excessively bent. In addition, even if the proximal end portion of the guidewire is excessively bent by a load that is received by the proximal end portion of the guidewire when the proximal end portion of the guidewire, for example, contacts vascular walls, it is less likely for permanent deformation to occur. Consequently, there is no possibility of subsequent operations being hindered. This makes it possible to continuously use the guidewire.

The guidewire according to the second aspect includes a second coil body and a first coil body that is wound around a distal end portion of the second coil body. In the guidewire, the second coil body includes a stranded wire that includes a plurality of wires that are twisted together. According to this guidewire, the wires can move slightly relative to each other over the entire second coil body in a longitudinal direction thereof. Therefore, it is possible to ensure a sufficient degree of freedom, increase flexibility, and ensure sufficient restoring force.

Therefore, for example, when inserting the guidewire along an inverted U-shaped winding path extending from a blood vessel in the right leg to a blood vessel in the left leg by a cross over method, it becomes possible to cause the entire guidewire to easily follow the shapes of blood vessels that are excessively bent. Even if the guidewire is excessively bent by a load that is received by the guidewire when the guidewire, for example, contacts vascular walls, it is possible to reduce permanent deformation over the entire guidewire in the longitudinal direction thereof. Consequently, there is no possibility of subsequent operations being hindered. This makes it possible to continuously use the guidewire.

### BRIEF DESCRIPTION OF THE DRAWINGS

FIG. 1 is a partial sectional enlarged view of a guidewire according to disclosed embodiments of the present invention.

FIG. 2 is a perspective view of a second coil body of the guidewire according to the disclosed embodiments of the present invention.

FIG. 3 is a partial sectional enlarged view of a guidewire according to disclosed embodiments of the present invention.

FIG. 4 is a partial sectional enlarged view of a guidewire according to disclosed embodiments of the present invention.

FIG. 5 is a partial sectional enlarged view of a guidewire according to disclosed embodiments of the present invention.

FIG. 6 is a partial sectional enlarged view of a guidewire according to disclosed embodiments of the present invention.

FIG. 7 is a perspective view of a second coil body of the guidewire according to the disclosed embodiments of the present invention.

FIG. 8 is a partial sectional enlarged view of a guidewire according to disclosed embodiments of the present invention.

FIG. 9 is a sectional view taken along line IX-IX of a second coil body in FIG. 8.

FIG. 10 is a partial sectional enlarged view of a guidewire according to disclosed embodiments of the present invention.

FIG. 11 is a sectional view taken along line XI-XI of a second coil body in FIG. 10.

FIG. 12 is a partial sectional enlarged view of a guidewire according to disclosed embodiments of the present invention.

FIG. 13 is a perspective view of a second coil body of the guidewire according to disclosed embodiments of the present invention.

FIG. 14 is a partial sectional enlarged view of a guidewire according to disclosed embodiments of the present invention.

FIG. 15 is a perspective view of a second coil body of the guidewire according to disclosed embodiments of the present invention.

FIG. 16 is a partial sectional enlarged view of a guidewire according to disclosed embodiments of the present invention.

FIG. 17 is a sectional view taken along line XVII-XVII of a second coil body in FIG. 16.

FIG. 18 is a partial sectional enlarged view of a guidewire according to disclosed embodiments of the present invention.

FIG. 19 is a sectional view taken along line XIX-XIX of a second coil body in FIG. 18.

### DETAILED DESCRIPTION OF EMBODIMENTS

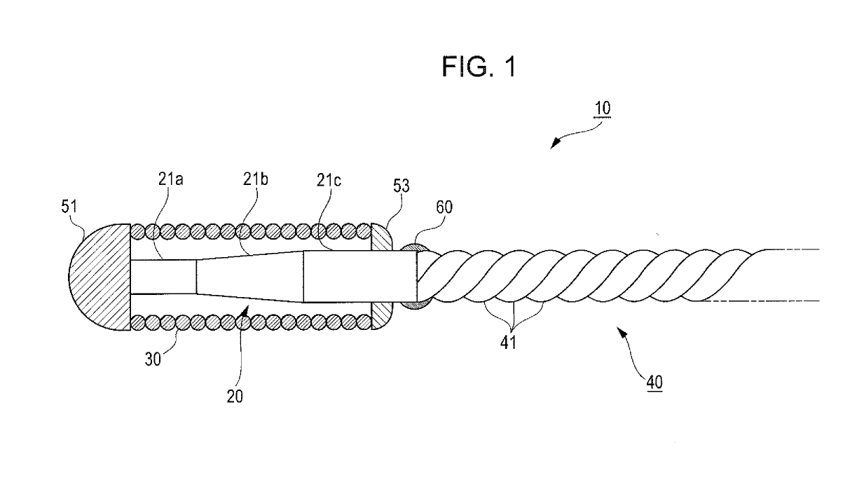

A guidewire 10 according to an embodiment of the present invention is described with reference to FIG. 1. In FIG. 1, the left side corresponds to a distal end that is inserted into a body and the right side corresponds to a proximal end that is operated by an operator, such as a doctor. Each figure schematically illustrates a guidewire, and its dimensional ratios differ from actual dimensional ratios.

The guidewire 10 shown in FIG. 1 is used, for example, for treating blood vessels of the lower limbs by a cross over method. The guidewire 10 includes a core shaft 20, a first coil body 30 that is wound around a distal end portion of the core shaft 20, and a second coil body 40 that is joined to a proximal end portion of the core shaft 20.

First, the core shaft 20 is described. From the distal end to the proximal end, the core shaft 20 includes a first linear portion 21a, a tapered portion 21b, and a second linear portion 21c. The first linear portion 21a is a portion at the tip of the core shaft 20 and is the most flexible portion of the core shaft 20. The first linear portion 21a is formed into a flat shape by pressing. The tapered portion 21b is circular in cross section. The diameter of the tapered portion 21b decreases towards the distal end. The diameter of the second linear portion 21c is larger than the diameter of the first linear portion 21a.

Materials of the core shaft 20 are not particularly limited to certain materials. Examples of materials include stainless steel (SUS304), a super-elastic alloy (such as a Ni—Ti alloy), a piano wire, and a cobalt-based alloy.

Next, the first coil body 30 is described. The first coil body 30 according to the FIG. 1 embodiment is a single-strand coil including spirally wound wires. However, the first coil body 30 is not limited to this type. For example, the first coil body 30 may be a multi-strand coil including stranded wires composed of a plurality of wires that are twisted together.

Materials of the first coil body 30 are not particularly limited to certain materials. Examples of materials include stainless steels (such as martensitic stainless steel, ferritic stainless steel, austenitic stainless steel, austenite two-phase stainless steel, ferrite two-phase stainless steel, and precipitation hardening stainless steel), super-elastic alloys (such as a Ni—Ti alloy), platinum, gold, tungsten, tantalum, and iridium (which are metals that are opaque to X rays), and alloys thereof.

As shown in FIG. 1, a distal end of the first coil body 30 is secured to a distal end of the core shaft 20 by a distal-end-side joint 51. A proximal end of the first coil body 30 is secured to the core shaft 20 by a proximal-end-side joint 53. Materials of the distal-end-side joint 51 and the proximal-end-side joint 53 are not limited to certain materials. Examples of materials include brazing metal materials such as a Sn—Pb alloy, a Pb—Ag alloy, a Sn—Ag alloy, and an Au—Sn alloy.

In FIG. 1, the proximal end portion of the core shaft 20 (that is, the proximal end of the second linear portion 21c) is exposed from the first coil body 30 (that is, is not covered by the first coil body 30). The second coil body 40 is joined to the proximal end of the core shaft 20 by a joint 60.

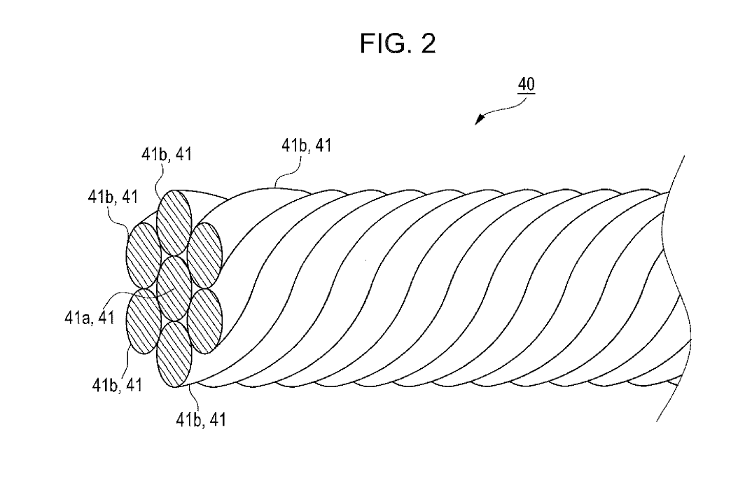

As shown in FIG. 2, the second coil body 40 includes a stranded wire composed of a plurality of wires 41 that are twisted together. More specifically, the second coil body 40 includes a core wire 41a and six side wires 41b that are wound so as to cover the outer periphery of the core wire 41a.

Materials of the second coil body 40 are not particularly limited to certain materials. Examples of materials include stainless steels (such as martensitic stainless steel, ferritic stainless steel, austenitic stainless steel, austenite two-phase stainless steel, ferrite two-phase stainless steel, and precipitation hardening stainless steel), super-elastic alloys (such as a Ni—Ti alloy), platinum, gold, tungsten, tantalum, and iridium (which are metals that are opaque to X rays), and alloys thereof.

Examples of materials of the joint 60, which joins the second coil body 40 to the proximal end of the core shaft 20, include brazing metal materials such as a Sn—Pb alloy, a Pb—Ag alloy, a Sn—Ag alloy, and an Au—Sn alloy. However, means for joining the second coil body 40 to the proximal end of the core shaft 20 is not particularly limited to brazing metal materials. Examples thereof include spot welding using laser and butt resistance welding such as butt seam welding.

In this way, in the FIG. 1 embodiment, the second coil body 40 including the stranded wire composed of the plurality of wires 41 that are twisted together is joined to the proximal end portion of the core shaft 20 by the joint 60. In such a stranded wire (the second coil body 40), the wires 41 can move slightly relative to each other. Therefore, in addition to the degree of freedom and the flexibility being high, sufficient restoring force is also ensured.

Therefore, for example, when inserting the guidewire 10 along an inverted U-shaped winding path extending from a blood vessel in the right leg to a blood vessel in the left leg by a cross over method, it becomes easy to follow the shapes of blood vessels that are excessively bent. In addition, even if the proximal end portion of the guidewire 10 is excessively bent by a load that is received by the proximal end portion of the guidewire 10 when the proximal end portion of the guidewire 10, for example, contacts vascular walls, it is less likely for permanent deformation to occur. Consequently, there is no possibility of subsequent operations being hindered. This makes it possible to continuously use the guidewire.

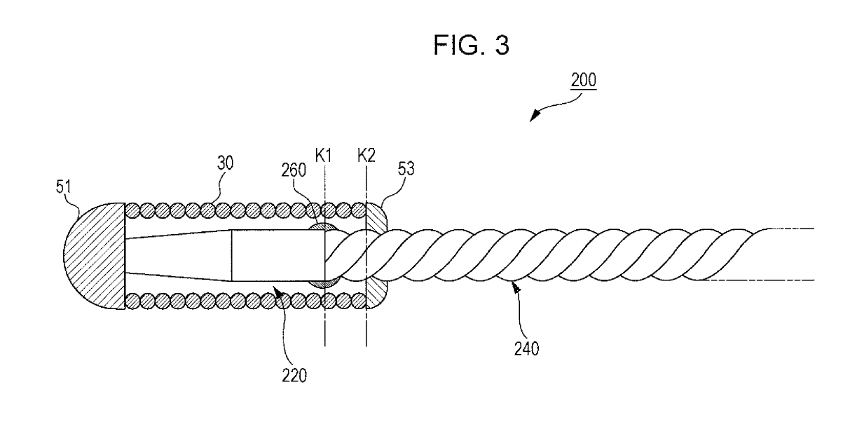

FIG. 3 is a partial sectional enlarged view of a guidewire 200 according to an embodiment of the present invention. In FIG. 3, the left side corresponds to a distal end that is inserted into a body and the right side corresponds to a proximal end that is operated by an operator, such as a doctor. Structural parts that correspond to those according to the above-described FIG. 1 embodiment are given the same reference numerals and are not described again. A description is hereunder given by focusing on the differences.

In the above-described FIG. 1 embodiment, the proximal end portion of the core shaft 20 is exposed beyond the first coil body 30 and the portion of the second coil body 40 that is joined to the core shaft 20 (the joint 60) is also exposed at the proximal end side of the first coil body 30. In contrast, in the guidewire 200 according to the FIG. 3 embodiment, a base end K1 of a core shaft 220 is positioned in the first coil body 30 (that is, closer to a distal end than a proximal end K2 of the first coil body 30) and a portion of a second coil body 240 that is joined to the core shaft 220 (that is, a joint 260) is provided in the first coil body 30.

According to this guidewire 200, as shown in FIG. 3, even if the joint 260 is provided in a convex manner at a surface of the core shaft 220 and a surface of the second coil body 240, the joint 260 is disposed in the first coil body 30 while being covered by the first coil body 30. Therefore, there is no possibility of vascular walls being damaged due to contact of the joint 260 with the vascular walls.

Since the portion of the second coil body 240 that is joined to the core shaft 220 (that is, the joint 260) is covered by the first coil body 30, the joint 260 does not contact a hard lesion in a blood vessel, so that it is possible to maintain the joining state between the core shaft 220 and the second coil body 240 in a good state.

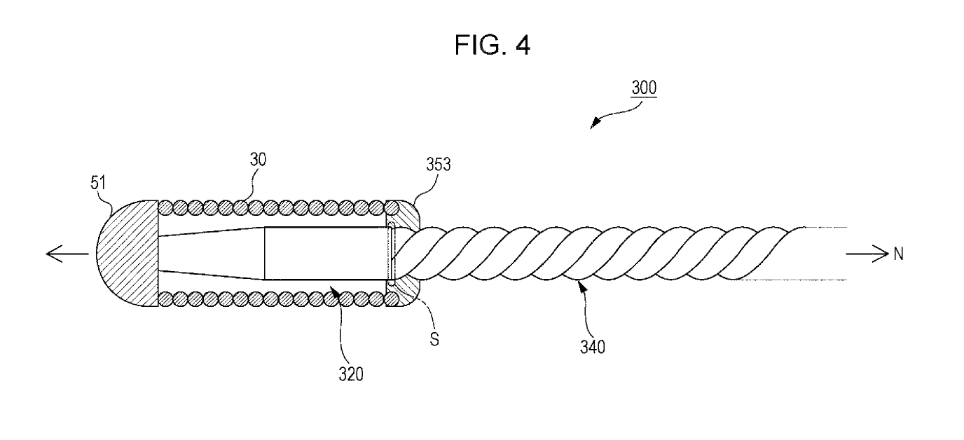

FIG. 4 is a partial sectional enlarged view of a guidewire 300 according to an embodiment of the present invention. In FIG. 4, the left side corresponds to a distal end that is inserted into a body and the right side corresponds to a proximal end that is operated by an operator, such as a doctor. Structural parts that correspond to those according to the above-described FIG. 3 embodiment are given the same reference numerals and are not described again. A description is hereunder given by focusing on the differences.

In the above-described FIG. 3 embodiment, as shown in FIG. 3, the proximal end of the core shaft 220 is positioned in the first coil body 30 (that is, closer to the distal end than the proximal end of the first coil body 30) and a portion of the second coil body 240 that is joined to the core shaft 220 (that is, the joint 260) is provided in the first coil body 30. In contrast, in the guidewire 300 according to the FIG. 4 embodiment, a proximal end of the first coil body 30 and a proximal end of the core shaft 320 are positioned at corresponding locations in a longitudinal direction N, and a proximal end joint 353 is provided so as to cover a joint S of a second coil body 340 that is joined to the core shaft 320.

That is, the proximal end joint 353 is provided so as to cover the entire periphery of the joint S of the second coil body 340 that is joined to the core shaft 320. In the FIG. 4 embodiment, this causes the joint S between the core shaft 320 and the second coil body 340 to be reinforced by the proximal end joint 353 where the proximal end of the first coil body 30 is joined to the core shaft 320.

As a result, for example, when inserting the guidewire 300 along an inverted U-shaped winding path extending from a blood vessel in the right leg to a blood vessel in the left leg by a cross over method, even if the guidewire 300 is largely bent by following such blood vessels, the second coil body 340 does not separate from the core shaft 320, so that it is possible to maintain the joining state of the second coil body 340 with respect to the core shaft 320 in a good state.

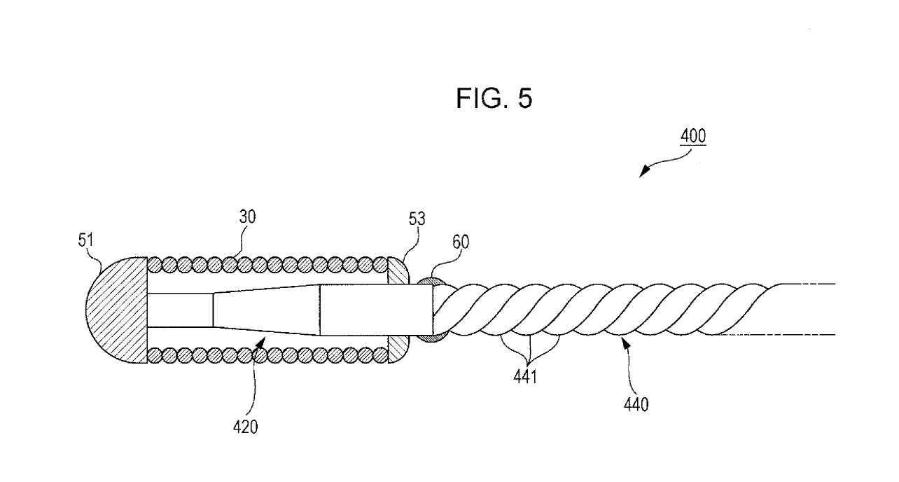

FIG. 5 is a partial sectional enlarged view of a guidewire 400 according to an embodiment of the present invention. In FIG. 5, the left side corresponds to a distal end that is inserted into a body and the right side corresponds to a proximal end that is operated by an operator, such as a doctor. Structural parts that correspond to those according to the above-described FIG. 1 embodiment are given the same reference numerals and are not described again. A description is hereunder given by focusing on the differences.

In the guidewire 400 according to the FIG. 5 embodiment, the material of wires 441 of a second coil body 440 is the same as the material of a core shaft 420. In the FIG. 5 embodiment, from the viewpoint of providing sufficient flexibility and restoring force with respect to bending, it is desirable that the material of the wires 441 of the second coil body 440 and the material of the core shaft 420 both be stainless steel.

Accordingly, by forming the wires 441 of the second coil body 440 out of the same material as the core shaft 420, the second coil body 440 is firmly joined with the core shaft 420, so that, during an operation, the second coil body 440 and the core shaft 420 are not separated from each other, as a result of which safety is ensured.

Although, in the FIG. 5 embodiment, an example using the guidewire according to the first embodiment is described, the structure according to the FIG. 5 embodiment may be used in each of the embodiments of FIGS. 3 and 4. Even in such cases, the operational advantages according to the FIG. 5 embodiment are not affected at all. Similarly, it is possible to maintain the joining state of the second coil body with respect to the core shaft in a good state.

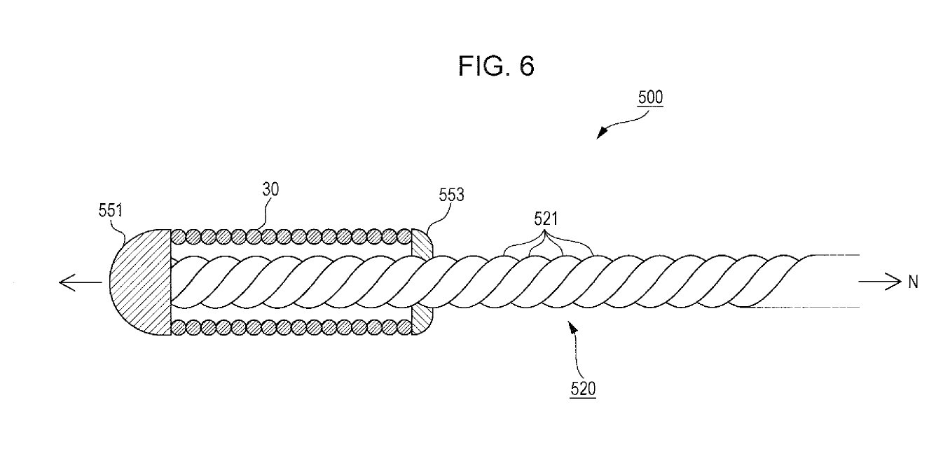

FIG. 6 is a partial sectional enlarged view of a guidewire 500 according to an embodiment of the present invention. In FIG. 6, the left side corresponds to a distal end that is inserted into a body and the right side corresponds to a proximal end that is operated by an operator, such as a doctor. Structural parts that correspond to those according to the above-described FIG. 1 embodiment are given the same reference numerals and are not described again. A description is hereunder given by focusing on the differences.

The guidewire 500 according to the FIG. 6 embodiment includes a second coil body 520 and a first coil body 30 that is wound around a distal end portion of the second coil body 520.

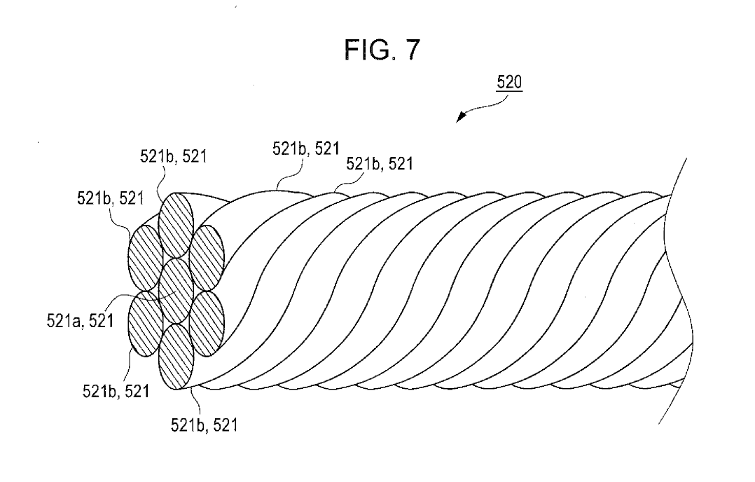

The second coil body 520 includes a stranded wire composed of a plurality of wires 521 that are twisted together over the entire second coil body 520 in a longitudinal direction N thereof. More specifically, similarly to the second coil body according to the above-described FIG. 1 embodiment, as shown in FIG. 7, the second coil body 520 includes a core wire 521a and six side wires 521b that are wound so as to cover the outer periphery of the core wire 521a.

A distal end of the coil body 30 is joined to a distal end of the second coil body 520 by a distal-end-side joint 551. A proximal end of the coil body 30 is joined to a substantially intermediate portion of the second coil body 520 by a proximal-end-side joint 553.

Accordingly, according to the guidewire 500 including the second coil body 520 including the stranded wire composed of the plurality of wires 521 that are twisted together over the entire guidewire 500 in the longitudinal direction N, the wires 521 can move slightly relative to each other over the entire second coil body 520 in the longitudinal direction thereof Therefore, it is possible to ensure a sufficient degree of freedom, increase flexibility, and ensure sufficient restoring force.

Consequently, for example, when inserting the guidewire 500 along an inverted U-shaped winding path extending from a blood vessel in the right leg to a blood vessel in the left leg by a cross over method, it becomes possible to cause the entire guidewire 500 to easily follow the shapes of blood vessels that are excessively bent. Even if the guidewire 500 is excessively bent by a load that is received by the guidewire 500 when the guidewire 500, for example, contacts vascular walls, it is possible to reduce permanent deformation over the entire guidewire 500 in the longitudinal direction N thereof. Consequently, there is no possibility of subsequent operations being hindered. This makes it possible to continuously use the guidewire 500.

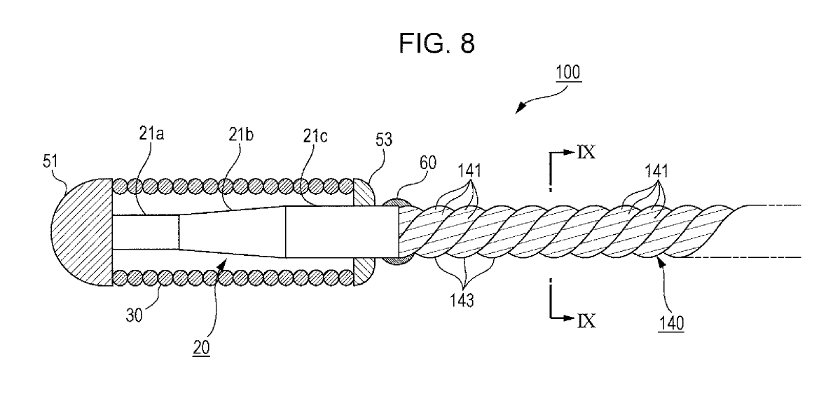

FIG. 8 is a partial sectional enlarged view of a guidewire 100 according to an embodiment of the present invention. In FIG. 8, the left side corresponds to a distal end that is inserted into a body and the right side corresponds to a proximal end that is operated by an operator, such as a doctor. Structural parts that correspond to those according to the above-described FIG. 1 embodiment are given the same reference numerals and are not described again. A description is hereunder given by focusing on the differences.

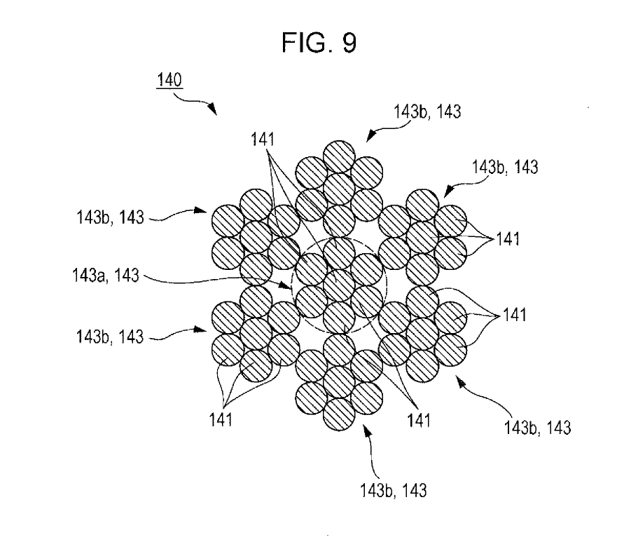

FIG. 9 is a sectional view taken along line IX-IX of a second coil body 140 in FIG. 8. FIG. 9 schematically illustrates the second coil body 140. The cross-sectional shape of stranded wires of the second coil body 140 has dimensional ratios that differ from actual dimensional ratios.

In the above-described FIG. 1 embodiment, the second coil body includes one stranded wire composed of a plurality of wires (six wires in the first embodiment) that are twisted together. In contrast, the guidewire 100 according to the FIG. 9 embodiment uses the second coil body 140 in which a plurality of stranded wires 143, each being composed of a plurality of wires 141 that are twisted together, are spirally wound.

More specifically, as shown in FIG. 9, the second coil body 140 includes a stranded wire 143a, which is a core member, and six stranded wires 143b, which are spirally wound so as to cover the outer periphery of the stranded wire 143a. The stranded wire 143a and the stranded wires 143b all have the same structure.

Materials of the second coil body 140 are not particularly limited to certain materials. Examples of materials include stainless steels (such as martensitic stainless steel, ferritic stainless steel, austenitic stainless steel, austenite two-phase stainless steel, ferrite two-phase stainless steel, and precipitation hardening stainless steel), super-elastic alloys (such as a Ni—Ti alloy), platinum, gold, tungsten, tantalum, and iridium (which are metals that are opaque to X rays), and alloys thereof.

Accordingly, in the guidewire 100 in which the second coil body 140 (which includes the stranded wires 143, each being composed of the plurality of wires 141 twisted together, that are spirally wound) is joined to a proximal end of the core shaft 20, in addition to making it possible for adjacent stranded wires 143 to move slightly relative to each other, it is also possible for the wires 141 of the stranded wires 143 to move slightly relative to each other. Therefore, compared to the above-described FIG. 1 embodiment, the degree of freedom is further increased and sufficient flexibility is ensured.

Consequently, for example, when inserting the guidewire 100 along an inverted U-shaped winding path extending from a blood vessel in the right leg to a blood vessel in the left leg by a cross over method, the guidewire 100 can very easily follow such blood vessels that are excessively bent, so that operability is increased.

Further, at the proximal end of the guidewire 100 (the second coil body 140), at the same time that the wires 141 are pressed together due to the application of rotational force, the stranded wires 143 are also pressed together, as a result of which contact pressure and thus contact therebetween is increased. As a result, sufficient torque is reliably transmitted towards the distal end and the guidewire 100 is pushed well into blood vessels in the legs that are excessively bent.

Although, in the FIG. 8 embodiment, an example using the guidewire according to the FIG. 1 embodiment is described, the structure according to the FIG. 8 embodiment may be used in each of the embodiments of FIGS. 3-5. Even in such cases, the operational advantages according to the FIG. 8 embodiment are not affected at all. Similarly, it is possible to ensure high operability of the guidewire.

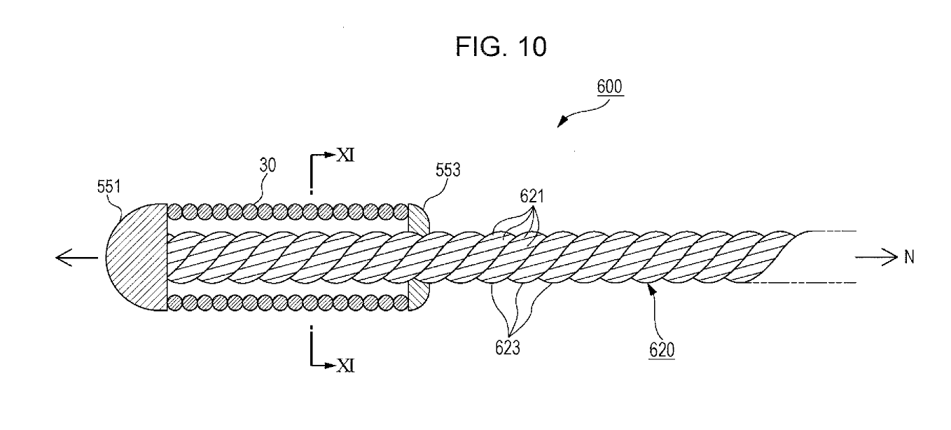

FIG. 10 is a partial sectional enlarged view of a guidewire 600 according to an embodiment of the present invention. In FIG. 10, the left side corresponds to a distal end that is inserted into a body and the right side corresponds to a proximal end that is operated by an operator, such as a doctor. Structural parts that correspond to those according to the above-described FIG. 6 embodiment are given the same reference numerals and are not described again. A description is hereunder given by focusing on the differences.

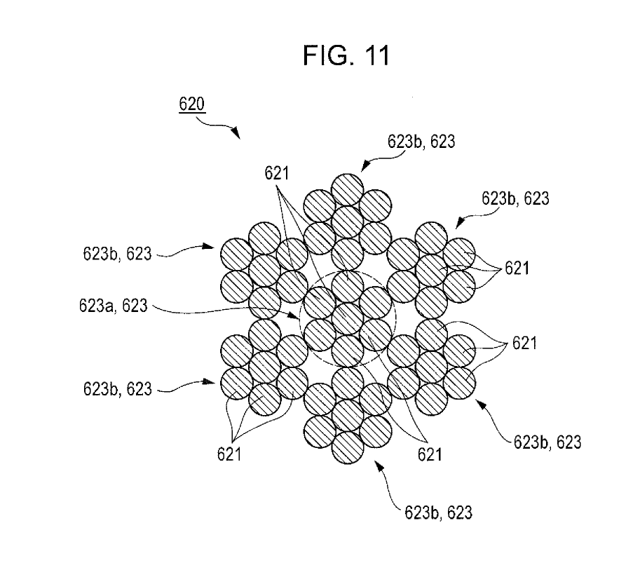

FIG. 11 is a sectional view taken along line XI-XI of a second coil body 620 in FIG. 10. FIG. 11 schematically illustrates the second coil body 620. The cross-sectional shape of stranded wires of the second coil body 620 has dimensional ratios that differ from actual dimensional ratios.

In the above-described FIG. 6 embodiment, the second coil body 520 includes one stranded wire composed of the plurality of wires 521 that are twisted together (refer to FIG. 6). In contrast, the guidewire 600 according to the FIG. 10 embodiment uses the second coil body 620 in which stranded wires 623, each being composed of a plurality of wires 621 that are twisted together, are spirally wound.

More specifically, as shown in FIG. 11, the second coil body 620 includes a stranded wire 623a, which is a core member, and six stranded wires 623b, which are spirally wound so as to cover the outer periphery of the stranded wire 623a. The stranded wire 623a and the stranded wires 623b all have the same structure.

Accordingly, according to the guidewire 600 including the second coil body 620 including the plurality of stranded wires 623 (each being composed of the plurality of wires 621 that are twisted together) that are spirally wound over the entire guidewire 600 in a longitudinal direction N, in addition to making it possible for adjacent stranded wires 623 to move slightly relative to each other, it is also possible for the wires 621 of the stranded wires 623 to move slightly relative to each other. Therefore, compared to the above-described FIG. 6 embodiment, the degree of freedom is further increased and sufficient flexibility is ensured.

Therefore, for example, when inserting the guidewire 600 along an inverted U-shaped winding path extending from a blood vessel in the right leg to a blood vessel in the left leg by a cross over method, it becomes possible to cause the entire guidewire 600 to more easily follow the shapes of blood vessels that are excessively bent. Even if the guidewire 600 is excessively bent by a load that is received by the guidewire 600 when the guidewire 600, for example, contacts vascular walls, it is possible to reliably reduce permanent deformation over the entire guidewire 600 in the longitudinal direction N thereof. Consequently, there is no possibility of subsequent operations being hindered. This makes it possible to continuously use the guidewire 600.

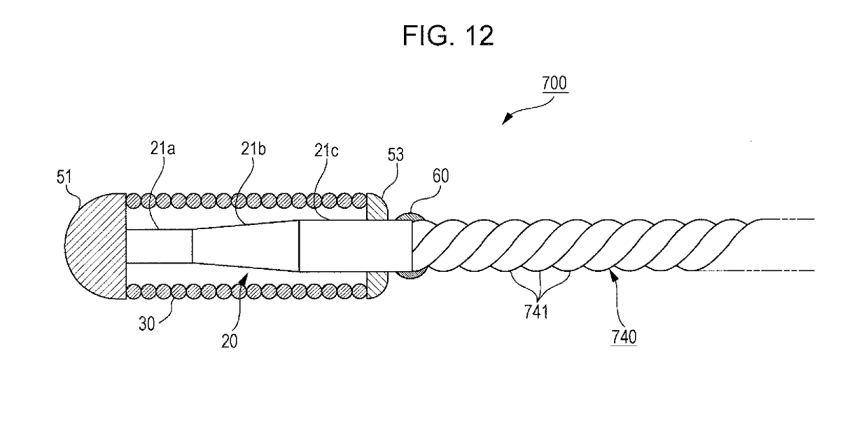

FIG. 12 is a partial sectional enlarged view of a guidewire 700 according to an embodiment of the present invention. In FIG. 12, the left side corresponds to a distal end that is inserted into a body and the right side corresponds to a proximal end that is operated by an operator, such as a doctor. Structural parts that correspond to those according to the above-described FIG. 1 embodiment are given the same reference numerals and are not described again. A description is hereunder given by focusing on the differences.

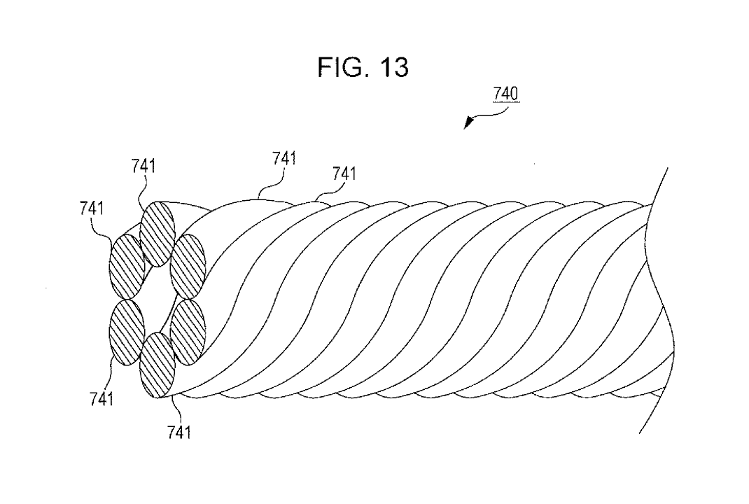

In the FIG. 1 embodiment, the second coil body 40 including the core wire 41a and six side wires 41b that are wound so as to cover the outer periphery of the core wire 41a is used (refer to FIG. 2). In contrast, in the guidewire 700 according to the FIG. 12 embodiment, as shown in FIG. 13, a second coil body 740 that has a hollow and that does not include a core wire is used. That is, the second coil body 740 according to the FIG. 12 embodiment includes a stranded wire composed of six wires 741 that are twisted together around a hollow.

According to the FIG. 12 embodiment, since a gap is provided in the center of the second coil body 740, compared to the FIG. 1 embodiment, the flexibility of the second coil body 740 is further increased. As a result, for example, when inserting the guidewire 700 along an inverted U-shaped winding path extending from a blood vessel in the right leg to a blood vessel in the left leg by a cross over method, it becomes possible to cause the guidewire 700 to reliably follow the blood vessels that are excessively bent. Consequently, the operability of the guidewire 700 is further increased.

Although, in the FIG. 12 embodiment, an example using the guidewire according to the FIG. 1 embodiment is described, the structure according to the FIG. 12 embodiment may be used in each of the embodiments of FIGS. 3-5. Even in such cases, the operational advantages according to the FIG. 12 embodiment are not affected at all. Similarly, it is possible to ensure high operability of the guidewire.

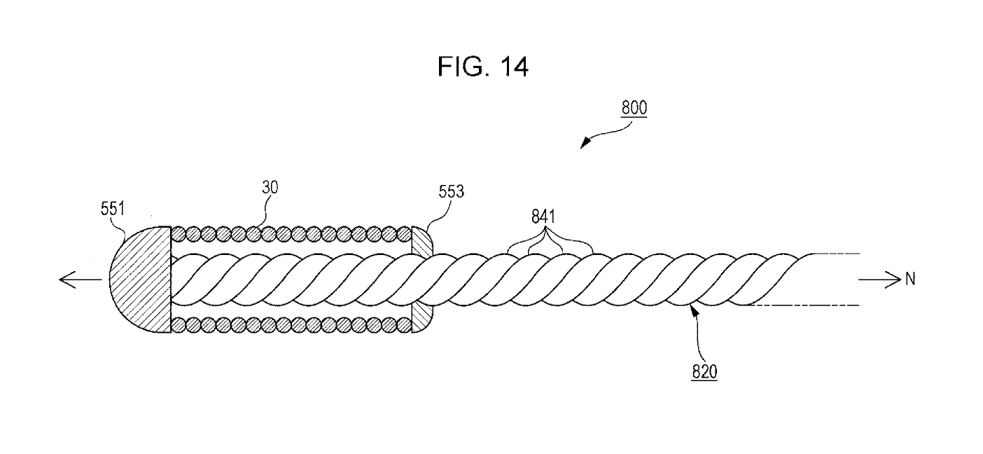

FIG. 14 is a partial sectional enlarged view of a guidewire 800 according to an embodiment of the present invention. In FIG. 14, the left side corresponds to a distal end that is inserted into a body and the right side corresponds to a proximal end that is operated by an operator, such as a doctor. Structural parts that correspond to those according to the above-described FIG. 6 embodiment are given the same reference numerals and are not described again. A description is hereunder given by focusing on the differences.

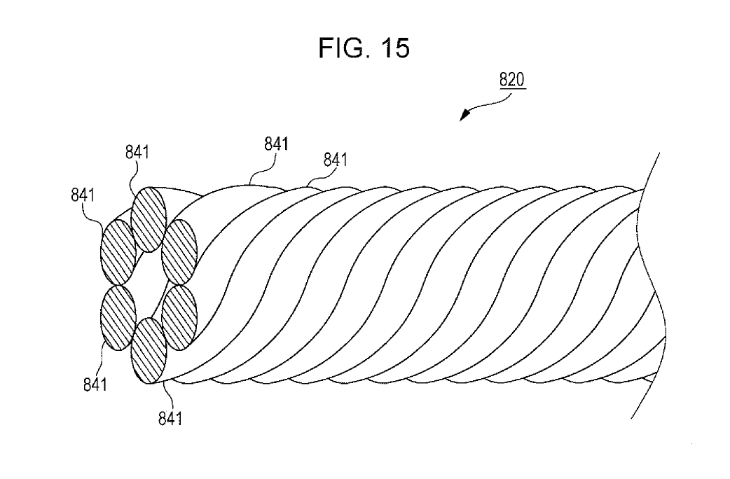

In the above-described FIG. 6 embodiment, the second coil body 520 including the core wire 521a and six side wires 521b that are wound so as to cover the outer periphery of the core wire 521a is used (refer to FIG. 7). In contrast, in the guidewire 800 according to the FIG. 14 embodiment, as shown in FIG. 15, a second coil body 820 that has a hollow and that does not include a core wire is used. That is, the second coil body 820 according to the FIG. 14 embodiment includes a stranded wire composed of six wires 841 that are twisted together around a hollow.

According to the FIG. 14 embodiment, since a gap is provided in the center of the second coil body 820, compared to the FIG. 6 embodiment, the flexibility of the second coil body 820 is further increased. That is, the guidewire 800 according to the FIG. 14 embodiment is provided with sufficient flexibility over the entire guidewire 800 in a longitudinal direction N thereof. As a result, for example, when inserting the guidewire 800 along an inverted U-shaped winding path extending from a blood vessel in the right leg to a blood vessel in the left leg by a cross over method, it becomes possible to cause the entire guidewire 800 to reliably follow the blood vessels that are excessively bent. Consequently, the operability of the guidewire 800 is further increased.

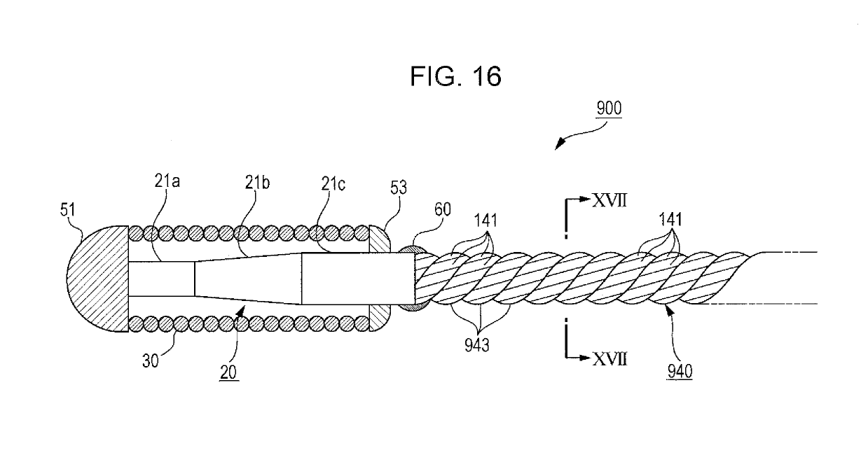

FIG. 16 is a partial sectional enlarged view of a guidewire 900 according to an embodiment of the present invention. In FIG. 16, the left side corresponds to a distal end that is inserted into a body and the right side corresponds to a proximal end that is operated by an operator, such as a doctor. Structural parts that correspond to those according to the above-described FIG. 8 embodiment are given the same reference numerals and are not described again. A description is hereunder given by focusing on the differences.

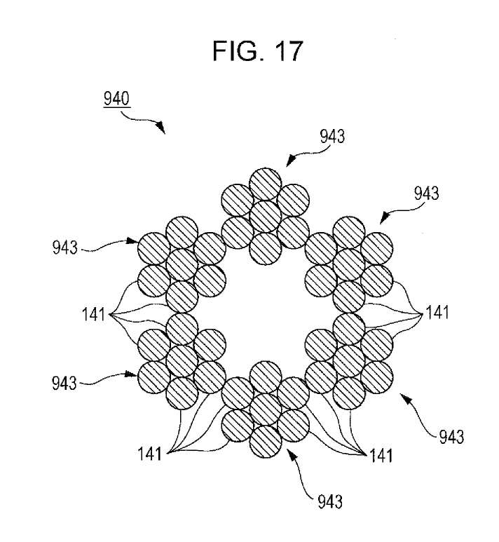

FIG. 17 is a sectional view taken along line XVII-XVII of a second coil body 940 in FIG. 16. FIG. 17 schematically illustrates the second coil body 940. The cross-sectional shape of stranded wires of the second coil body 940 has dimensional ratios that differ from actual dimensional ratios.

In the above-described FIG. 8 embodiment, the second coil body 140 including the stranded wire 143a, which is a core wire, and six stranded wires 143b, which are spirally wound so as to cover the outer periphery of the stranded wire 143a, is used (refer to FIG. 9). In contrast, in the guidewire 900 according to the FIG. 16 embodiment, as shown in FIG. 17, the second coil body 940 that has a hollow and that does not include a core member at the center is used. That is, in the FIG. 16 embodiment, the second coil body 940 having a hollow and including six stranded wires 943 that are twisted together is used.

According to the FIG. 16 embodiment, since a gap is provided in the center of the second coil body 940, compared to the FIG. 8 embodiment, the flexibility of the second coil body 940 is further increased. As a result, for example, when inserting the guidewire 900 along an inverted U-shaped winding path extending from a blood vessel in the right leg to a blood vessel in the left leg by a cross over method, it becomes possible to cause the guidewire 900 to more reliably follow the blood vessels that are excessively bent. Consequently, the operability of the guidewire 900 is further increased.

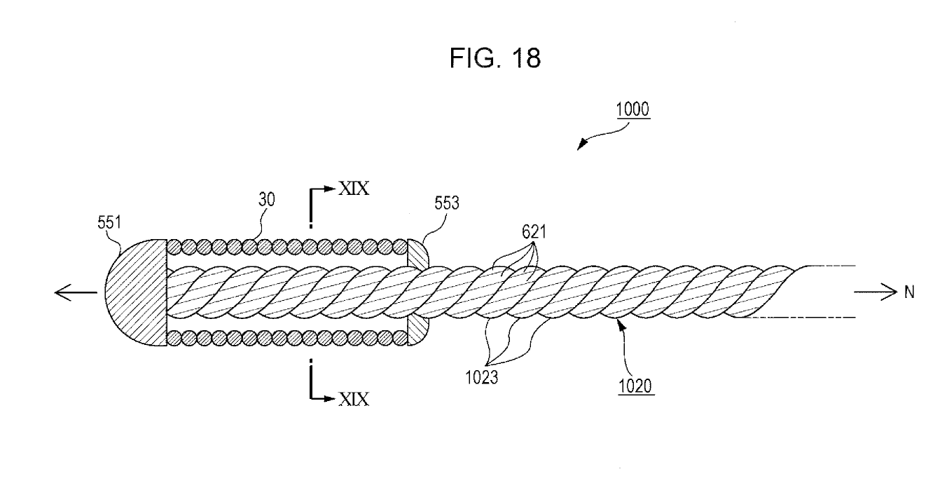

FIG. 18 is a partial sectional enlarged view of a guidewire 1000 according to an embodiment of the present invention. In FIG. 18, the left side corresponds to a distal end that is inserted into a body and the right side corresponds to a proximal end that is operated by an operator, such as a doctor. Structural parts that correspond to those according to the above-described FIG. 10 embodiment are given the same reference numerals and are not described again. A description is hereunder given by focusing on the differences.

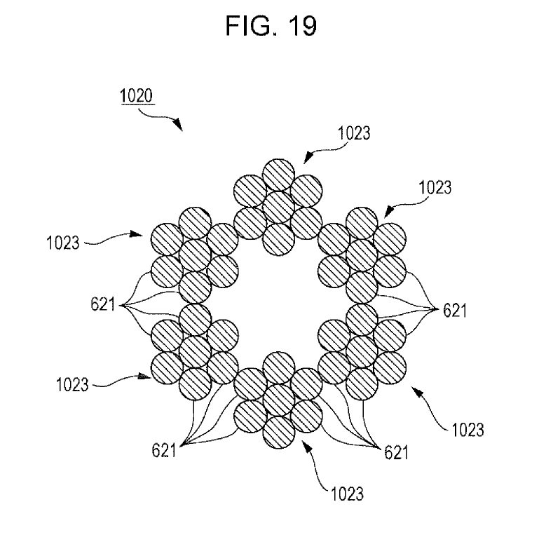

FIG. 19 is a sectional view taken along line D-D of a second coil body 1020 in FIG. 18. FIG. 19 schematically illustrates the second coil body 1020. The cross-sectional shape of stranded wires of the second coil body 1020 has dimensional ratios that differ from actual dimensional ratios.

In the above-described FIG. 10 embodiment, the second coil body 620 including the stranded wire 623a, which is a core wire, and six stranded wires 623b, which are spirally wound so as to cover the outer periphery of the stranded wire 623a, is used (refer to FIG. 11). In contrast, in the guidewire 1000 according to the FIG. 18 embodiment, as shown in FIG. 19, the second coil body 1020 that has a hollow and that does not include a core member is used. That is, in the FIG. 18 embodiment, the second coil body 1020 having a hollow and including six stranded wires 1023 that are twisted together is used.

According to the FIG. 18 embodiment, since a gap is provided in the center of the second coil body 1020, compared to the above-described FIG. 10 embodiment, the flexibility of the second coil body 1020 is further increased. That is, the guidewire 1000 according to the FIG. 18 embodiment is provided with sufficient flexibility over the entire guidewire 1000 in a longitudinal direction N thereof. As a result, for example, when inserting the guidewire 1000 along an inverted U-shaped winding path extending from a blood vessel in the right leg to a blood vessel in the left leg by a cross over method, it becomes possible to cause the entire guidewire 1000 to reliably flexibly follow the blood vessels that are excessively bent. Consequently, the operability of the guidewire 1000 is further increased.

What is claimed is:

1. A guidewire comprising:
a core shaft;
a first coil body that is wound around a distal end portion of the core shaft; and
a second coil body joined to a proximal end portion of the core shaft by a joint, the second coil body including a stranded wire that includes a plurality of wires that are twisted together.

2. The guidewire according to claim 1, wherein a proximal end of the first coil body is located proximally of the joint such that the joint is provided within the first coil body.

3. The guidewire according to claim 1, wherein a material of the second coil body is identical to a material of the core shaft.

4. The guidewire according to claim 1, wherein the second coil body includes a plurality of spirally wound stranded wires, each stranded wire including a plurality of wires that are twisted together.

5. The guidewire according to claim 1, wherein the second coil body is a hollow body.

6. The guidewire according to claim 4, wherein the second coil body is a hollow body.

7. The guidewire according to claim 1, further comprising a proximal end joint that joins a proximal end of the first coil body to the core shaft, the proximal end joint contacting the joint by which the second coil body is joined to the proximal end portion of the core shaft.

8. A guidewire comprising:
a second coil body; and
a first coil body that is wound around a distal end portion of the second coil body,
wherein the second coil body includes a stranded wire that includes a plurality of wires that are twisted together.

9. The guidewire according to claim 8, wherein the second coil body includes a plurality of spirally wound stranded wires, each stranded wire being composed of a plurality of wires that are twisted together.

10. The guidewire according to claim 8, wherein the second coil body is a hollow body.

11. The guidewire according to claim 9, wherein the second coil body is a hollow body.

12. The guidewire according to claim 8, further comprising a distal end joint that attaches a distal end of the first coil body to a distal end of the second coil body.

13. The guidewire according to claim 12, further comprising a proximal end joint that attaches a proximal end of the first coil body to an intermediate portion of the second coil body, the intermediate portion of the second coil body being located between the distal end of the second coil body and a proximal end of the second coil body.
